(19) United States
(12) Patent Application Publication
(10) Pub. No.: US 2024/0331146 A1
(43) Pub. Date: Oct. 3, 2024

(54) IMAGE PROCESSING APPARATUS, IMAGE PROCESSING METHOD, IMAGE PROCESSING PROGRAM, LEARNING APPARATUS, LEARNING METHOD, AND LEARNING PROGRAM

(71) Applicant: FUJIFILM CORPORATION, Tokyo (JP)

(72) Inventor: Nobuyuki HIRAHARA, Tokyo (JP)

(73) Assignee: FUJIFILM CORPORATION, Tokyo (JP)

(21) Appl. No.: 18/596,653

(22) Filed: Mar. 6, 2024

(30) Foreign Application Priority Data
Mar. 28, 2023 (JP) 2023-051616

(51) Int. Cl. G06T 7/00; G06T 5/50; G06T 7/11; G16H 30/40

(52) U.S. Cl. CPC G06T 7/0012; G06T 5/50; G06T 7/11; G16H 30/40; G06T 2207/20221; G06T 2207/30096

(57) ABSTRACT

An image processing apparatus generates an estimated medical image in which at least one partial region as an estimation target among a plurality of partial regions in an anatomical region included in a medical image is estimated based on at least one partial region other than the estimation target.

### CROSS-REFERENCE TO RELATED APPLICATIONS

This application claims priority from Japanese Patent Application No. 2023-051616, filed on Mar. 28, 2023, the entire disclosure of which is incorporated herein by reference.

### BACKGROUND

### 1. Technical Field

The present disclosure relates to an image processing apparatus, an image processing method, an image processing program, a learning apparatus, a learning method, and a learning program.

### 2. Description of the Related Art

JP2006-325937A discloses a technique of detecting a candidate region of an abnormal shadow from a medical image and setting a small region including at least a part of the detected candidate region as a region-of-interest. In this technique, the small region existing in the vicinity of the region-of-interest is set as a vicinity region, and an artificial image in which the region-of-interest and the vicinity region are normal is generated.

### SUMMARY

In diagnosis of a lesion such as a pancreatic cancer, for example, a medical image interpreter may determine whether or not the lesion has occurred based on an abnormality such as a shape change and a property change of a peripheral portion of the lesion due to occurrence of the lesion in the medical image. In this case, in a case in which a medical image in which an abnormality such as a shape change or a property change has not occurred can be accurately generated, it is possible to effectively support an interpretation of the interpreter.

The present disclosure has been made in view of the above circumstances, and an object of the present disclosure is to provide an image processing apparatus, an image processing method, an image processing program, a learning apparatus, a learning method, and a learning program capable of accurately generating a medical image in which an abnormality has not occurred.

According to a first aspect, there is provided an image processing apparatus comprising: at least one processor, in which the processor generates an estimated medical image in which at least one partial region as an estimation target among a plurality of partial regions in an anatomical region included in a medical image is estimated based on at least one partial region other than the estimation target.

A second aspect provides the image processing apparatus according to the first aspect, in which the processor divides the anatomical region into the plurality of partial regions.

A third aspect provides the image processing apparatus according to the first aspect or the second aspect, in which the processor performs control of displaying the partial region as the estimation target in the estimated medical image and a region corresponding to the partial region as the estimation target in the medical image in a comparable manner.

A fourth aspect provides the image processing apparatus according to an one of the first aspect to the third aspect, in which the processor performs control of displaying information indicating a difference between the partial region as the estimation target in the estimated medical image and a region corresponding to the partial region as the estimation target in the medical image.

A fifth aspect provides the image processing apparatus according to the third aspect or the fourth aspect, in which the processor performs the control in a case in which a value indicating a difference between the partial region as the estimation target in the estimated medical image and the region corresponding to the partial region as the estimation target in the medical image is equal to or greater than a threshold value.

A sixth aspect provides the image processing apparatus according to the fifth aspect, in which the processor generates the estimated medical image for each of the plurality of partial regions, and performs the control in a case in which a value indicating the difference for at least one estimated medical image is equal to or greater than the threshold value.

A seventh aspect provides the image processing apparatus according to any one of the first aspect to the sixth aspect, in which the estimated medical image is an image in which the estimated medical image generated for at least one of the plurality of partial regions is combined with the anatomical region in the medical image.

An eighth aspect provides the image processing apparatus according to any one of the first aspect to the seventh aspect, in which the processor performs a process of detecting a candidate for an abnormality in the anatomical region, and generates the estimated medical image using only a trained model corresponding to the partial region in which the detected candidate for the abnormality exists among a plurality of trained models that are respectively trained in advance for the plurality of partial regions, the trained model being used to generate the estimated medical image.

A ninth aspect provides the image processing apparatus according to any one of the first aspect to the eighth aspect, in which the anatomical region is a pancreas, and the plurality of partial regions include a head part, a body part, and a tail part.

According to a tenth aspect, there is provided an image processing method executed by a processor of an image processing apparatus, the method comprising: generating an estimated medical image in which at least one partial region as an estimation target among a plurality of partial regions in an anatomical region included in a medical image is estimated based on at least one partial region other than the estimation target.

According to an eleventh aspect, there is provided an image processing program for causing a processor of an image processing apparatus to execute: generating an estimated medical image in which at least one partial region as an estimation target among a plurality of partial regions in an anatomical region included in a medical image is estimated based on at least one partial region other than the estimation target.

According to a twelfth aspect, there is provided a learning apparatus comprising: at least one processor, in which the processor performs machine learning using an estimated medical image in which at least one first partial region as an estimation target among a plurality of partial regions in an anatomical region included in a medical image is estimated based on at least one second partial region other than the first partial region, and a normal medical image in which an abnormality has not occurred in the anatomical region, as learning data, thereby generating a trained model that outputs the estimated medical image in response to an input of the second partial region.

According to a thirteenth aspect, there is provided a learning method executed by a processor of a learning apparatus, the method comprising: performing machine learning using an estimated medical image in which at least one first partial region as an estimation target among a plurality of partial regions in an anatomical region included in a medical image is estimated based on at least one second partial region other than the first partial region, and a normal medical image in which an abnormality has not occurred in the anatomical region, as learning data, thereby generating a trained model that outputs the estimated medical image in response to an input of the second partial region.

According to a fourteenth aspect, there is provided a learning program for causing a processor of a learning apparatus to execute: performing machine learning using an estimated medical image in which at least one first partial region as an estimation target among a plurality of partial regions in an anatomical region included in a medical image is estimated based on at least one second partial region other than the first partial region, and a normal medical image in which an abnormality has not occurred in the anatomical region, as learning data, thereby generating a trained model that outputs the estimated medical image in response to an input of the second partial region.

According to the present disclosure, it is possible to accurately generate a medical image in which an abnormality has not occurred.

### BRIEF DESCRIPTION OF THE DRAWINGS

FIG. 1 is a block diagram showing a schematic configuration of a medical information system.

FIG. 2 is a block diagram showing an example of a hardware configuration of an image processing apparatus according to a first embodiment.

FIG. 3 is a block diagram showing an example of a functional configuration of the image processing apparatus in a learning phase.

FIG. 4 is a diagram for describing a first trained model.

FIG. 5 is a diagram for describing a second trained model.

FIG. 6 is a diagram for describing a third trained model.

FIG. 7 is a block diagram showing an example of a functional configuration of the image processing apparatus in an operation phase according to the first embodiment.

FIG. 8 is a diagram showing an example of a display screen.

FIG. 9 is a diagram showing an example of a display screen according to a modification example.

FIG. 10 is a flowchart showing an example of a learning process.

FIG. 11 is a flowchart showing an example of a diagnosis support process according to the first embodiment.

FIG. 12 is a block diagram showing an example of a hardware configuration of an image processing apparatus according to a second embodiment.

FIG. 13 is a block diagram showing an example of a functional configuration of the image processing apparatus in an operation phase according to the second embodiment.

FIG. 14 is a diagram for describing a process of generating a medical image using a trained model according to the second embodiment.

FIG. 15 is a flowchart showing an example of a diagnosis support process according to the second embodiment.

FIG. 16 is a diagram for describing a trained model according to a modification example.

FIG. 17 is a diagram for describing a trained model according to a modification example.

FIG. 18 is a diagram for describing a trained model according to a modification example.

FIG. 19 is a diagram for describing a trained model according to a modification example.

FIG. 20 is a diagram showing an example of a display screen according to a modification example.

FIG. 21 is a diagram showing an example of a display screen according to a modification example.

### DETAILED DESCRIPTION

Hereinafter, examples of an embodiment for implementing the technique of the present disclosure will be described in detail with reference to the drawings.

### First Embodiment

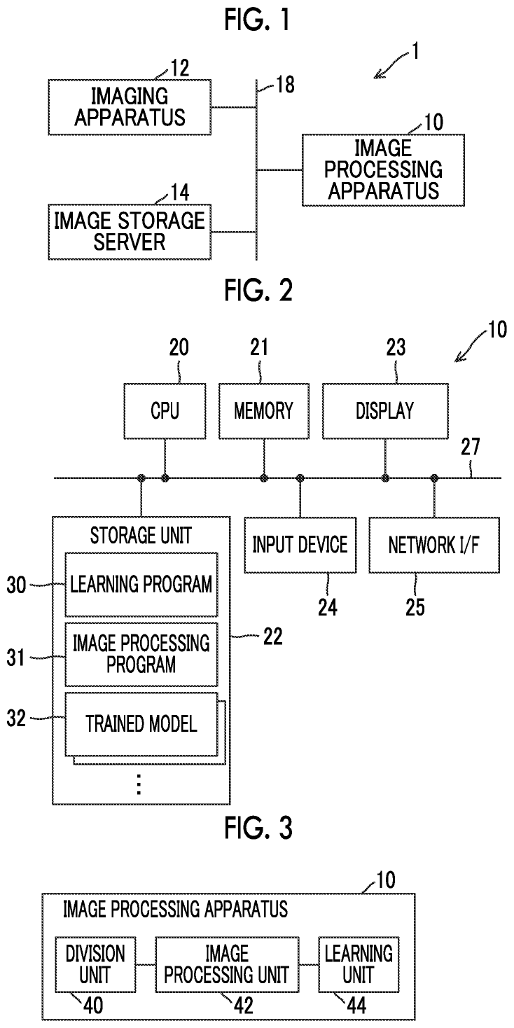

First, a configuration of a medical information system 1 according to the present embodiment will be described with reference to FIG. 1. As shown in FIG. 1, the medical information system 1 includes an image processing apparatus 10, an imaging apparatus 12, and an image storage server 14. The image processing apparatus 10, the imaging apparatus 12, and the image storage server 14 are connected to each other in a communicable manner via a wired or wireless network 18. The image processing apparatus 10 is, for example, a computer such as a personal computer or a server computer.

The imaging apparatus 12 is an apparatus that generates a medical image showing a diagnosis target part of a subject by imaging the part. Examples of the imaging apparatus 12 include a simple X-ray imaging apparatus, an endoscope apparatus, a computed tomography (CT) apparatus, a magnetic resonance imaging (MRI) apparatus, and a positron emission tomography (PET) apparatus. In the present embodiment, an example will be described in which the imaging apparatus 12 is a CT device and the diagnosis target part is an abdomen. That is, the imaging apparatus 12 according to the present embodiment generates a CT image of the abdomen of the subject as a three-dimensional medical image formed of a plurality of tomographic images. The medical image generated by the imaging apparatus 12 is transmitted to the image storage server 14 via the network 18 and stored by the image storage server 14.

The image storage server 14 is a computer that stores and manages various types of data, and comprises a large-capacity external storage device and database management software. The image storage server 14 receives the medical image generated by the imaging apparatus 12 via the network 18, and stores and manages the received medical image. A storage format of image data by the image storage server 14 and the communication with another device via the network 18 are based on a protocol such as digital imaging and communication in medicine (DICOM).

Next, a hardware configuration of the image processing apparatus 10 according to the present embodiment will be described with reference to FIG. 2. As shown in FIG. 2, the image processing apparatus 10 includes a central processing unit (CPU) 20, a memory 21 as a temporary storage region, and a non-volatile storage unit 22. In addition, the image processing apparatus 10 includes a display 23 such as a liquid crystal display, an input device 24 such as a keyboard and a mouse, and a network interface (I/F) 25 that is connected to the network 18. The CPU 20, the memory 21, the storage unit 22, the display 23, the input device 24, and the network I/F 25 are connected to a bus 27. The CPU 20 is an example of a processor according to the technique of the present disclosure.

The storage unit 22 is realized by a hard disk drive (HDD), a solid state drive (SSD), a flash memory, or the like. A learning program 30 and an image processing program 31 are stored in the storage unit 22 as a storage medium. The CPU 20 reads out the learning program 30 from the storage unit 22, then expands the learning program 30 in the memory 21, and executes the expanded learning program 30. In addition, the CPU 20 reads out the image processing program 31 from the storage unit 22, expands the image processing program 31 in the memory 21, and executes the expanded image processing program 31.

Incidentally, in a case in which an abnormality has occurred in a partial region in an anatomical region in a medical image, it is possible to effectively support interpretation of the medical image by an interpreter in a case in which a medical image in which a partial region in which the abnormality has not occurred is estimated can be generated. The image processing apparatus 10 according to the present embodiment has a function of generating a medical image in which a state in which an abnormality has not occurred is estimated, in order to effectively support the interpretation of the medical image by the interpreter. In the present embodiment, an example in which the pancreas is applied as the anatomical region to be processed will be described.

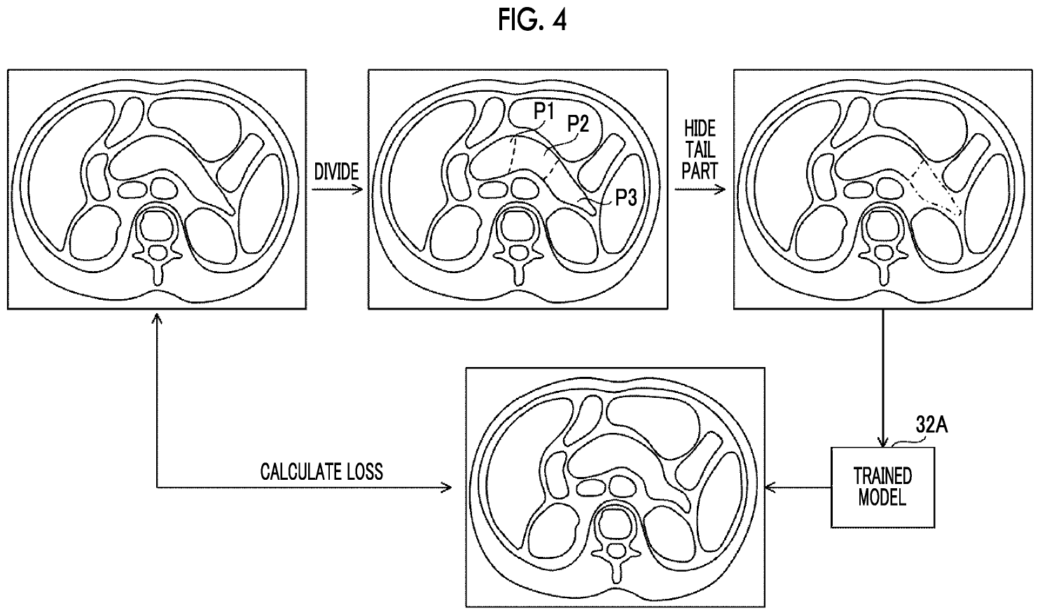

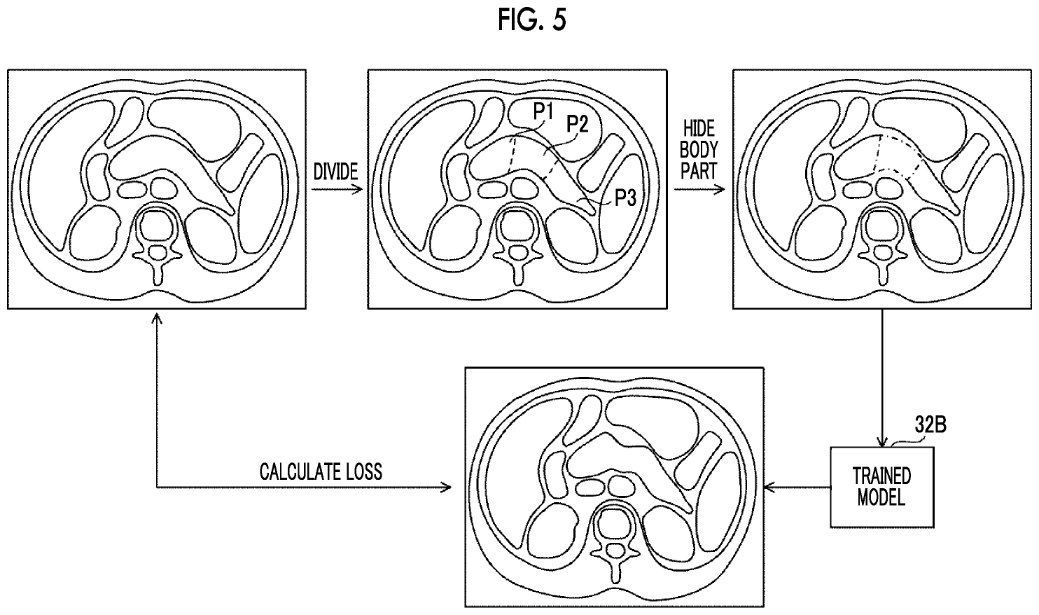

In order to realize the above-described function, a plurality of trained models 32 are stored in the storage unit 22. In the present embodiment, a case in which the number of the trained models 32 is three will be described. With reference to FIG. 3, a functional configuration of the image processing apparatus 10 in a learning phase of the trained model 32 according to the present embodiment will be described. In the following, in a case of distinguishing between the three trained models 32, A, B, and C are assigned to ends as in trained models 32A, 32B, and 32C. The image processing apparatus 10 in the learning phase is an example of a learning apparatus according to the disclosed technique.

As shown in FIG. 3, the image processing apparatus 10 in the learning phase includes a division unit 40, an image processing unit 42, and a learning unit 44. The CPU 20 executes the learning program 30 to function as the division unit 40, the image processing unit 42, and the learning unit 44.

As shown in FIGS. 4 to 6, the division unit 40 divides the pancreas as an example of the anatomical region included in the medical image into a plurality of partial regions. In the present embodiment, the division unit 40 divides the pancreas into three partial regions of a head part P1, a body part P2, and a tail part P3. For this division, for example, boundaries of the head part P1, the body part P2, and the tail part P3, which are defined in medical texts, such as a vein and an artery, are used. In addition, for the division, for example, statistical values such as average values of values indicating sizes such as diameters and volumes of the head part P1, the body part P2, and the tail part P3 in the medical images obtained by imaging the pancreas of a large number of patients may be used.

The medical image used in the learning phase is a medical image in which no abnormality has occurred in the pancreas, that is, a medical image in which the pancreas is in a healthy state (hereinafter, referred to as a “normal medical image”). The abnormality of the pancreas in the present embodiment includes not only a lesion that is a target to be directly treated, such as cancer, a cyst, and inflammation, but also an indirect finding. The indirect finding means a feature of at least one of a shape or a property of peripheral tissue of the lesion associated with occurrence of the lesion. For example, examples of the indirect finding suspected to be a pancreatic cancer include a shape abnormality such as partial atrophy and swelling in the pancreas.

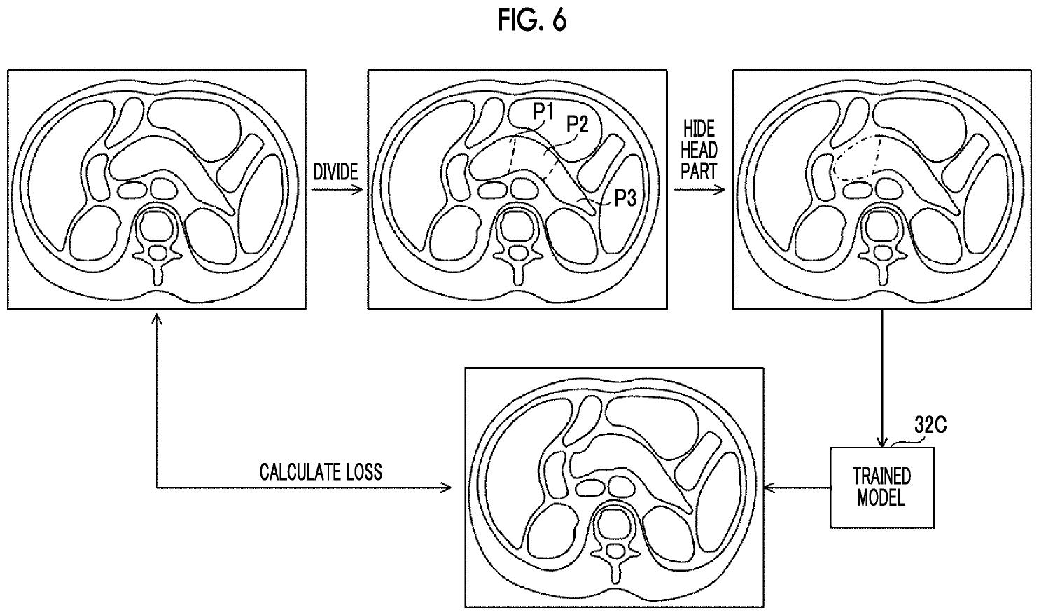

As shown in FIG. 4, the image processing unit 42 executes image processing to hide the tail part P3 in the medical image. In addition, as shown in FIG. 5, the image processing unit 42 executes image processing to hide the body part P2 in the medical image. In addition, as shown in FIG. 6, the image processing unit 42 executes image processing to hide the head part P1 in the medical image. Examples of the image processing include a process of filling a region to be hidden with a predetermined color such as a background color. In the examples of FIGS. 4 to 6, a contour of the region hidden by the image processing is indicated by a one-dot chain line.

The learning unit 44 performs machine learning using an estimated medical image in which at least one first partial region as an estimation target among a plurality of partial regions in the pancreas included in the medical image is estimated based on at least one second partial region other than the first partial region, and the normal medical image, as learning data (which may be referred to as “teacher data”). As a result, the learning unit 44 generates a trained model 32 that outputs the estimated medical image in response to an input of the second partial region.

Specifically, as shown in FIG. 4, the learning unit 44 inputs the medical image after the execution of the image processing to hide the tail part P3 by the image processing unit 42 to the trained model 32A. The trained model 32A generates and outputs an estimated medical image in which the tail part P3 is estimated based on the head part P1 and the body part P2, which are two partial regions in the pancreas included in the input medical image. The learning unit 44 calculates a loss between the normal medical image before the execution of the division processing by the division unit 40 and the estimated medical image output from the trained model 32A, and trains the trained model 32A such that the loss is minimized. The trained model 32A is a model that generates an estimated medical image in which the tail part P3 of the pancreas in the input medical image is estimated based on the head part P1 and the body part P2 of the pancreas in the medical image.

In addition, as shown in FIG. 5, the learning unit 44 inputs the medical image after the execution of the image processing to hide the body part P2 by the image processing unit 42 to the trained model 32B. The trained model 32B generates and outputs an estimated medical image in which the body part P2 is estimated based on the head part P1 and the tail part P3, which are two partial regions in the pancreas included in the input medical image. The learning unit 44 calculates a loss between the normal medical image before the execution of the division processing by the division unit 40 and the estimated medical image output from the trained model 32B, and trains the trained model 32B such that the loss is minimized. The trained model 32B is a model that generates an estimated medical image in which the body part P2 of the pancreas in the input medical image is estimated based on the head part P1 and the tail part P3 of the pancreas in the medical image.

In addition, as shown in FIG. 6, the learning unit 44 inputs the medical image after the execution of the image processing to hide the head part P1 by the image processing unit 42 to the trained model 32C. The trained model 32C generates and outputs an estimated medical image in which the head part P1 is estimated based on the body part P2 and the tail part P3, which are two partial regions in the pancreas included in the input medical image. The learning unit 44 calculates a loss between the normal medical image before the execution of the division processing by the division unit 40 and the estimated medical image output from the trained model 32C, and trains the trained model 32C such that the loss is minimized. The trained model 32C is a model that generates an estimated medical image in which the head part P1 of the pancreas in the input medical image is estimated based on the body part P2 and the tail part P3 of the pancreas in the medical image.

Each of the three trained models 32 is configured by, for example, a convolutional neural network (CNN). The learning unit 44 performs the above learning using a large number of medical images. The three trained models 32 trained as described above are stored in the storage unit 22. As described above, since the medical images in which the pancreas is in a healthy state are used for the learning of the three trained models 32, the medical images in which the healthy states of the head part P1, the body part P2, and the tail part P3 are estimated are output from the three trained models 32.

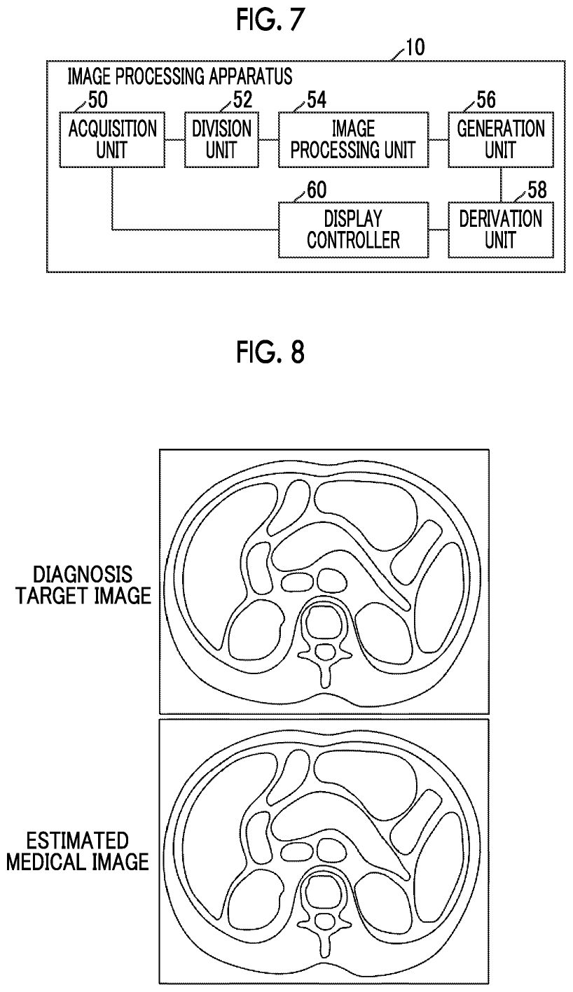

Next, with reference to FIG. 7, a functional configuration of the image processing apparatus 10 in an operation phase of the trained model 32 according to the present embodiment will be described. As shown in FIG. 7, the image processing apparatus 10 includes an acquisition unit 50, a division unit 52, an image processing unit 54, a generation unit 56, a derivation unit 58, and a display controller 60. The CPU 20 executes the image processing program 31 to function as the acquisition unit 50, the division unit 52, the image processing unit 54, the generation unit 56, the derivation unit 58, and the display controller 60.

The acquisition unit 50 acquires a medical image to be diagnosed (hereinafter, referred to as a “diagnosis target image”) from the image storage server 14 via the network I/F 25. As with the division unit 40, the division unit 52 divides the pancreas as an example of the anatomical region included in the diagnosis target image into three partial regions of the head part P1, the body part P2, and the tail part P3.

The image processing unit 54 executes image processing to hide the tail part P3 in the diagnosis target image, as with the image processing unit 42. In addition, the image processing unit 54 executes image processing to hide the body part P2 in the diagnosis target image. In addition, the image processing unit 54 executes image processing to hide the head part P1 in the diagnosis target image.

The generation unit 56 generates an estimated medical image in which at least one partial region as an estimation target among a plurality of partial regions in the anatomical region included in the diagnosis target image is estimated based on at least one partial region other than the estimation target. In the present embodiment, the generation unit 56 generates the estimated medical image for each of the plurality of partial regions. The estimated medical image is an image in which the estimated medical image generated for at least one of the plurality of partial regions is combined with the anatomical region in the diagnosis target image.

Specifically, the generation unit 56 inputs the diagnosis target image after the execution of the image processing to hide the tail part P3 by the image processing unit 54 to the trained model 32A. The trained model 32A generates and outputs an estimated medical image in which the tail part P3 is estimated based on the head part P1 and the body part P2, which are two partial regions in the pancreas included in the input diagnosis target image. As described above, the generation unit 56 generates an estimated medical image (hereinafter, referred to as a “tail part estimated medical image”) in which the tail part P3 among the head part P1, the body part P2, and the tail part P3 in the pancreas included in the diagnosis target image is estimated based on the head part P1 and the body part P2.

In addition, the generation unit 56 inputs the diagnosis target image after the execution of the image processing to hide the body part P2 by the image processing unit 54 to the trained model 32B. The trained model 32B generates and outputs an estimated medical image in which the body part P2 is estimated based on the head part P1 and the tail part P3, which are two partial regions in the pancreas included in the input diagnosis target image. As described above, the generation unit 56 generates an estimated medical image (hereinafter, referred to as a “body part estimated medical image”) in which the body part P2 among the head part P1, the body part P2, and the tail part P3 in the pancreas included in the diagnosis target image is estimated based on the head part P1 and the tail part P3.

In addition, the generation unit 56 inputs the diagnosis target image after the execution of the image processing to hide the head part P1 by the image processing unit 54 to the trained model 32C. The trained model 32C generates and outputs an estimated medical image in which the head part P1 is estimated based on the body part P2 and the tail part P3, which are two partial regions in the pancreas included in the input diagnosis target image. As described above, the generation unit 56 generates an estimated medical image (hereinafter, referred to as a “head part estimated medical image”) in which the head part P1 among the head part P1, the body part P2, and the tail part P3 in the pancreas included in the diagnosis target image is estimated based on the body part P2 and the tail part P3.

The derivation unit 58 derives a value indicating a difference between the partial region as the estimation target in the estimated medical image and a region corresponding to the partial region as the estimation target in the diagnosis target image for each of the estimated medical images generated by the generation unit 56. That is, the derivation unit 58 derives a value indicating a difference between the tail part P3 in the tail part estimated medical image and the tail part P3 in the diagnosis target image. In addition, the derivation unit 58 derives a value indicating a difference between the body part P2 in the body part estimated medical image and the body part P2 in the diagnosis target image. In addition, the derivation unit 58 derives a value indicating a difference between the head part P1 in the head part estimated medical image and the head part P1 in the diagnosis target image. As an example of the value indicating the difference of the partial region, a value indicating a difference in size of the partial region, such as a difference in volume, a difference in major axis, and a difference in cross-sectional area is used. In addition, as an example of the value indicating the difference of the partial region, a difference in a statistical value such as an average value, a variance, and a total value of the CT values of the partial region is also used.

In a case in which a value indicating the difference derived by the derivation unit 58 for at least one estimated medical image is equal to or greater than a threshold value, the display controller 60 performs control of displaying the partial region as the estimation target in the estimated medical image, of which the value indicating the difference is equal to or greater than the threshold value, and the partial region as the estimation target in the diagnosis target image, in a comparable manner. In the present embodiment, as shown in FIG. 8 as an example, the display controller 60 performs control of displaying the estimated medical image and the diagnosis target image side by side on the display 23 to display the images in a comparable manner. In the example of FIG. 8, a case in which a value indicating a difference between the tail part P3 in the tail part estimated medical image and the tail part P3 in the diagnosis target image is equal to or greater than the threshold value is shown.

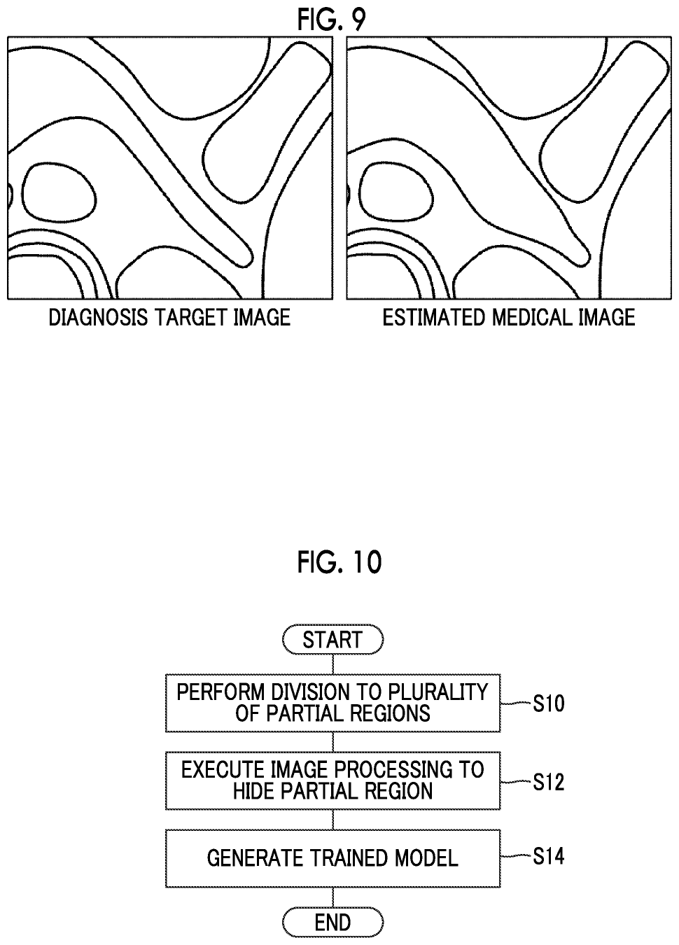

As shown in FIG. 9, the display controller 60 may perform control of displaying the partial region in the estimated medical image of which the value indicating the difference is equal to or greater than the threshold value and the partial region as the estimation target in the diagnosis target image in an enlarged state side by side on the display 23 to display the partial regions in a comparable manner. In the example of FIG. 9, a value indicating a difference between the tail part P3 in the tail part estimated medical image and the tail part P3 in the diagnosis target image is equal to or greater than the threshold value, and the tail part P3 is shown in an enlarged state.

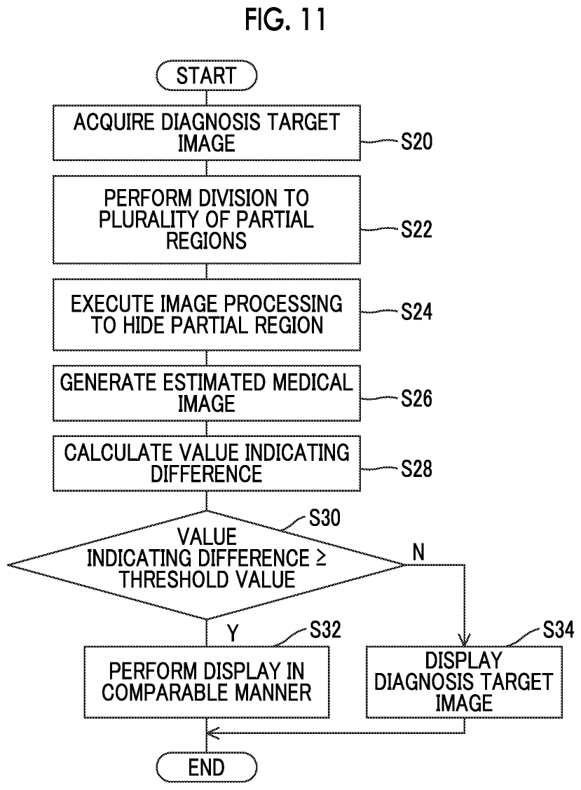

Next, an operation of the image processing apparatus 10 according to the present embodiment will be described with reference to FIGS. 10 and 11. The CPU 20 executes the learning program 30, whereby a learning process shown in FIG. 10 is executed. The CPU 20 executes the image processing program 31 to execute a diagnosis support process shown in FIG. 11. The learning process shown in FIG. 10 and the diagnosis support process shown in FIG. 11 are executed, for example, in a case in which an instruction to start an execution is input by a user.

In step S10 of FIG. 10, as described above, the division unit 40 divides the pancreas into three partial regions of the head part P1, the body part P2, and the tail part P3. In step S12, the image processing unit 42 executes image processing to hide the tail part P3 in the medical image. In addition, the image processing unit 42 executes image processing to hide the body part P2 in the medical image. In addition, the image processing unit 42 executes image processing to hide the head part P1 in the medical image.

In step S14, as described above, the learning unit 44 generates the trained model 32A by performing machine learning using the medical image after the execution of the image processing to hide the tail part P3 in step S12 and the normal medical image as learning data. In addition, as described above, the learning unit 44 generates the trained model 32B by performing machine learning using the medical image after the execution of the image processing to hide the body part P2 in step S12 and the normal medical image as learning data. In addition, as described above, the learning unit 44 generates the trained model 32C by performing machine learning using the medical image after the execution of the image processing to hide the head part P1 in step S12 and the normal medical image as learning data. Then, the learning unit 44 performs control of storing the generated trained models 32A, 32B, and 32C in the storage unit 22. In a case in which the process of step S14 ends, the learning process ends.

In step S20 of FIG. 11, the acquisition unit 50 acquires the diagnosis target image from the image storage server 14 via the network I/F 25. In step S22, as described above, the division unit 52 divides the pancreas included in the diagnosis target image acquired in step S20 into three partial regions of the head part P1, the body part P2, and the tail part P3. In step S24, the image processing unit 54 executes image processing to hide the tail part P3 in the diagnosis target image. In addition, the image processing unit 54 executes image processing to hide the body part P2 in the diagnosis target image. In addition, the image processing unit 54 executes image processing to hide the head part P1 in the diagnosis target image.

In step S26, the generation unit 56 generates the tail part estimated medical image by inputting the diagnosis target image after the execution of the image processing to hide the tail part P3 in step S24 to the trained model 32A. In addition, the generation unit 56 generates the body part estimated medical image by inputting the diagnosis target image after the execution of the image processing to hide the body part P2 in step S24 to the trained model 32B. In addition, the generation unit 56 generates the head part estimated medical image by inputting the diagnosis target image after the execution of the image processing to hide the head part P1 in step S24 to the trained model 32C.

In step S28, the derivation unit 58 derives a value indicating a difference between the tail part P3 in the tail part estimated medical image generated in step S26 and the tail part P3 in the diagnosis target image. In addition, the derivation unit 58 derives a value indicating a difference between the body part P2 in the body part estimated medical image generated in step S26 and the body part P2 in the diagnosis target image. In addition, the derivation unit 58 derives a value indicating a difference between the head part P1 in the head part estimated medical image generated in step S26 and the head part P1 in the diagnosis target image.

In step S30, the display controller 60 determines whether or not at least one of the values indicating the differences derived for the three estimated medical images in step S28 is equal to or greater than a threshold value. In a case in which an affirmative determination is made in the determination, the process proceeds to step S32. In step S32, as described above, the display controller 60 performs control of displaying the partial region in the estimated medical image of which the value indicating the difference is equal to or greater than the threshold value and the partial region as the estimation target in the diagnosis target image in a comparable manner. In a case in which the process of step S32 ends, the diagnosis support process ends.

On the other hand, in a case where a negative determination is made in the determination in step S30, the process proceeds to step S34. In step S34, the display controller 60 performs control of displaying the diagnosis target image on the display 23. In a case in which the process of step S34 ends, the diagnosis support process ends.

As described above, according to the present embodiment, it is possible to accurately generate a medical image in which an abnormality such as a shape change or a property change has not occurred, and as a result, it is possible to effectively support the interpretation of the medical image by the interpreter.

### Second Embodiment

A second embodiment of the disclosed technique will be described. A configuration of the medical information system 1 according to the present embodiment is the same as that of the first embodiment, and thus the description thereof will be omitted.

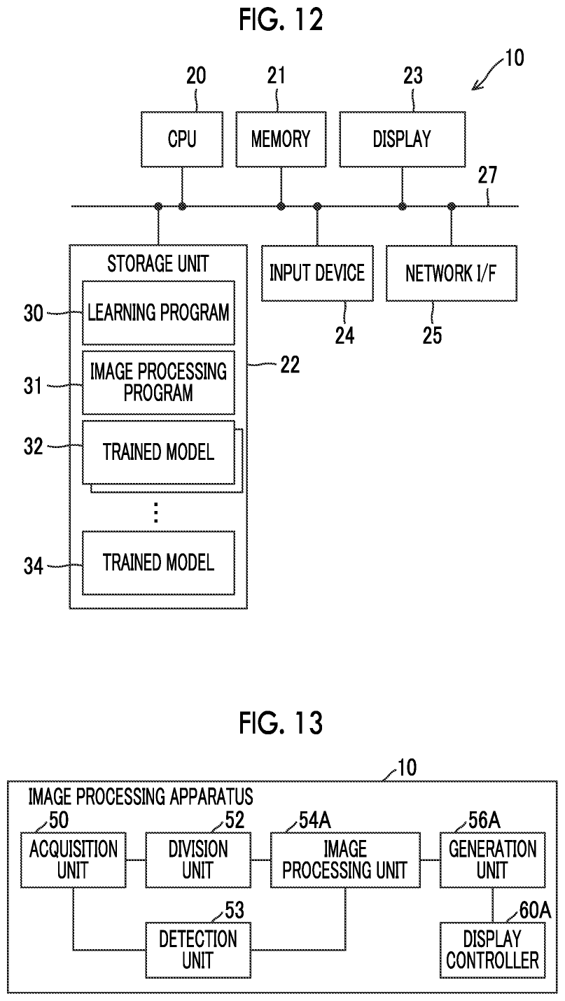

A hardware configuration of the image processing apparatus 10 according to the present embodiment will be described with reference to FIG. 12. The same components as those of the image processing apparatus 10 according to the first embodiment are denoted by the same reference numerals, and the description thereof will be omitted. As shown in FIG. 12, a trained model 34 is further stored in the storage unit 22 of the image processing apparatus 10 according to the present embodiment.

The trained model 34 is a model for detecting a candidate for an abnormality such as a lesion and an indirect finding from the medical image. The trained model 34 is configured by, for example, a CNN. The trained model 34 is a model that is trained through machine learning using, for example, a large number of combinations of a medical image including an abnormality and information specifying a region in which the abnormality exists in the medical image as learning data.

Next, a functional configuration of the image processing apparatus 10 according to the present embodiment will be described. A functional configuration of the image processing apparatus 10 in the learning phase of the trained model 32 is the same as that of the first embodiment, and thus the description thereof will be omitted.

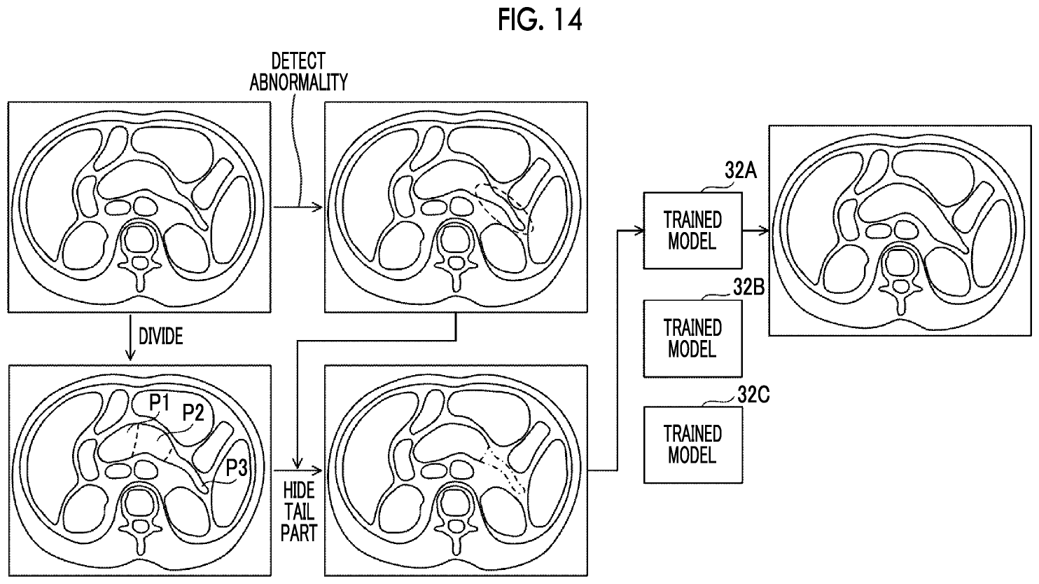

With reference to FIG. 13, a functional configuration of the image processing apparatus 10 in an operation phase of the trained model 32 according to the present embodiment will be described. A functional unit having the same function as the image processing apparatus 10 in the operation phase of the trained model 32 according to the first embodiment is denoted by the same reference numeral, and the description thereof will be omitted. As shown in FIG. 13, the image processing apparatus 10 includes an acquisition unit 50, a division unit 52, a detection unit 53, an image processing unit 54A, a generation unit 56A, and a display controller 60A. The CPU 20 executes the image processing program 31 to function as the acquisition unit 50, the division unit 52, the detection unit 53, the image processing unit 54A, the generation unit 56A, and the display controller 60A.

As shown in FIG. 14, the detection unit 53 performs a process of detecting a candidate for an abnormality in the pancreas as an example of the anatomical region included in the diagnosis target image. Specifically, the detection unit 53 inputs the diagnosis target image to the trained model 34. The trained model 34 detects a candidate for an abnormality in the pancreas included in the input diagnosis target image, and outputs information specifying a region in which the detected candidate for an abnormality exists. This information may be, for example, an image in which a specific value is stored in a voxel of a region in which a candidate for an abnormality exists in the diagnosis target image, or information indicating any of three partial regions of the head part P1, the body part P2, and the tail part P3 in which a candidate for an abnormality exists.

The image processing unit 54A executes image processing to hide a partial region in which the candidate for the abnormality detected by the detection unit 53 exists. The generation unit 56A generates an estimated medical image using only the trained model 32 corresponding to the partial region in which the candidate for the abnormality detected by the detection unit 53 exists among the plurality of trained models 32. Specifically, the generation unit 56A generates an estimated medical image by inputting the diagnosis target image after the execution of the image processing to hide the partial region in which the candidate for the abnormality exists by the image processing unit 54A to the trained model 32 corresponding to the partial region in which the candidate for the abnormality exists. FIG. 14 shows an example in which a candidate for an abnormality (for example, atrophy of the tail part P3) is detected in the tail part P3 surrounded by a broken line, the image processing to hide the tail part P3 is executed, and the diagnosis target image after the execution of the image processing is input to the trained model 32A to generate a tail part estimated medical image.

The display controller 60A performs control of displaying the partial region in which the candidate for the abnormality exists in the estimated medical image generated by the generation unit 56A and the partial region as the estimation target in the diagnosis target image in a comparable manner. The specific content of the control of performing the display in a comparable manner is the same as that of the first embodiment, and thus the description thereof will be omitted.

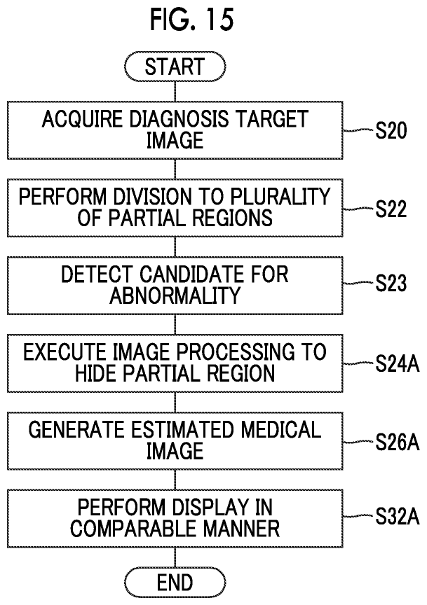

Next, an operation of the image processing apparatus 10 according to the present embodiment will be described with reference to FIG. 15. The learning process according to the present embodiment is the same as the learning process (see FIG. 10) according to the first embodiment, and thus the description thereof will be omitted. The CPU 20 executes the image processing program 31 to execute a diagnosis support process shown in FIG. 15. The diagnosis support process shown in FIG. 15 is executed, for example, in a case in which an instruction to start an execution is input by the user. Steps in FIG. 15 for executing the same process as FIG. 11 are be designated by the same step numbers, and thus the description thereof will be omitted.

In a case in which the process in step S22 in FIG. 15 ends, the process proceeds to step S23. In step S23, as described above, the detection unit 53 performs a process of detecting a candidate for an abnormality in the pancreas included in the diagnosis target image. In step S24A, the image processing unit 54A executes the image processing to hide the partial region in which the candidate for the abnormality detected in step S23 exists.

In step S26A, as described above, the generation unit 56A generates an estimated medical image using only the trained model 32 corresponding to the partial region in which the candidate for the abnormality detected in step S23 exists among the plurality of trained models 32. In step S32A, the display controller 60A performs control of displaying the partial region in which the candidate for the abnormality exists in the estimated medical image generated in step S26A and the partial region as the estimation target in the diagnosis target image in a comparable manner. In a case in which the process of step S32A ends, the diagnosis support process ends.

As described above, according to the present embodiment, the same effect as the first embodiment can be accomplished.

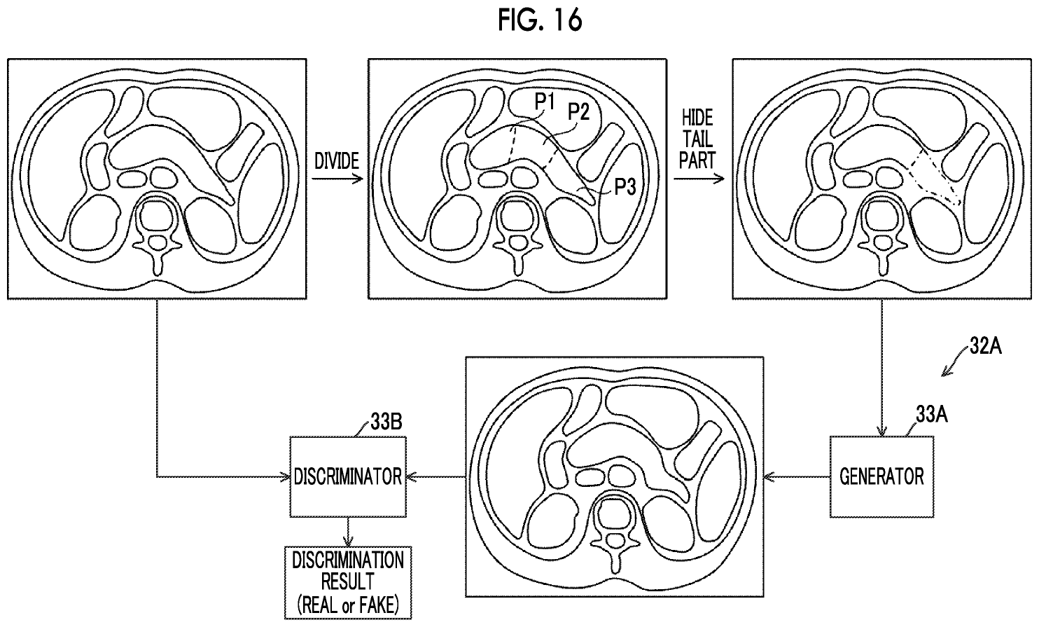

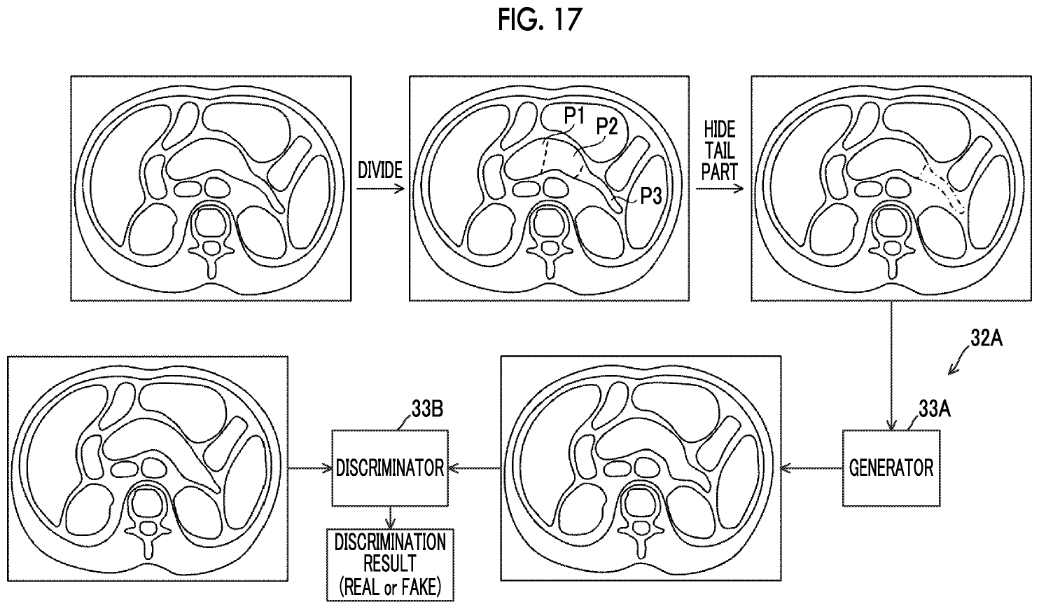

In each of the above embodiments, as the trained model 32, a generative model called a generative adversarial network (GAN) may be applied. FIG. 16 shows an example of the trained model 32A that generates an estimated medical image in which the tail part P3 of the pancreas in the medical image in this embodiment example is estimated based on the head part P1 and the body part P2 of the pancreas in the medical image. As shown in FIG. 16, the trained model 32A in this embodiment example includes a generator 33A and a discriminator 33B. Each of the generator 33A and the discriminator 33B is configured by, for example, a CNN.

The learning unit 44 inputs the medical image after the execution of the image processing to hide the tail part P3 by the image processing unit 42 to the generator 33A. The generator 33A generates and outputs an estimated medical image in which the tail part P3 is estimated based on the head part P1 and the body part P2, which are two partial regions in the pancreas included in the input medical image. The discriminator 33B discriminates whether the estimated medical image is a real medical image or a fake medical image by comparing the medical image before the execution of the division processing by the division unit 40 with the estimated medical image output from the generator 33A. Then, the discriminator 33B outputs information indicating whether the estimated medical image is a real medical image or a fake medical image as a discrimination result. As the discrimination result, a probability that the estimated medical image is a real medical image is used. In addition, as the discrimination result, two values such as “1” indicating that the estimated medical image is a real medical image and “0” indicating that the generated medical image is a fake medical image are used.

The learning unit 44 trains the generator 33A such that the generator 33A can generate an estimated medical image closer to a real medical image. In addition, the learning unit 44 trains the discriminator 33B such that the discriminator 33B can more accurately discriminate whether the estimated medical image is a fake medical image. For example, the learning unit 44 uses a loss function in which a loss of the discriminator 33B increases as a loss of the generator 33A decreases to perform learning such that the loss of the generator 33A is minimized in training the generator 33A. In addition, the learning unit 44 uses the loss function to perform learning such that the loss of the discriminator 33B is minimized in training the discriminator 33B. The trained model 32 is a model obtained by alternately training the generator 33A and the discriminator 33B using a large amount of learning data. In this embodiment example, a loss called a reconstruction loss between the medical image before the execution of the division processing by the division unit 40 and the estimated medical image may be further used for learning.

In FIG. 16, an example in which the GAN is applied to the trained model 32A has been described, but the GAN can be similarly applied to the trained models 32B and 32C.

In addition, as shown in FIG. 17, the medical image input to the generator 33A may be based on a medical image in which an abnormality has occurred in the pancreas. FIG. 17 shows an example in which atrophy occurs in the tail part P3. In this embodiment example, the image processing unit 42 executes image processing to hide the tail part P3 in which an abnormality has occurred. The learning unit 44 inputs the medical image after the execution of the image processing to hide the tail part P3 by the image processing unit 42 to the generator 33A. The discriminator 33B discriminates whether the estimated medical image is a real medical image or a fake medical image by comparing the medical image in which an abnormality has not occurred in the pancreas with the estimated medical image output from the generator 33A. The medical image in which an abnormality has not occurred in the pancreas and the medical image in which an abnormality has occurred in the pancreas may be obtained by imaging the same patient or may be obtained by imaging different patients.

In FIG. 17, an example in which the trained model 32A is trained based on the medical image in which an abnormality has occurred in the tail part P3 has been described, but the same learning can be applied to the trained models 32B and 32C. In this case, a medical image in which an abnormality has occurred in the body part P2 is used for the learning of the trained model 32B, and a medical image in which an abnormality has occurred in the head part P1 is used for the learning of the trained model 32C.

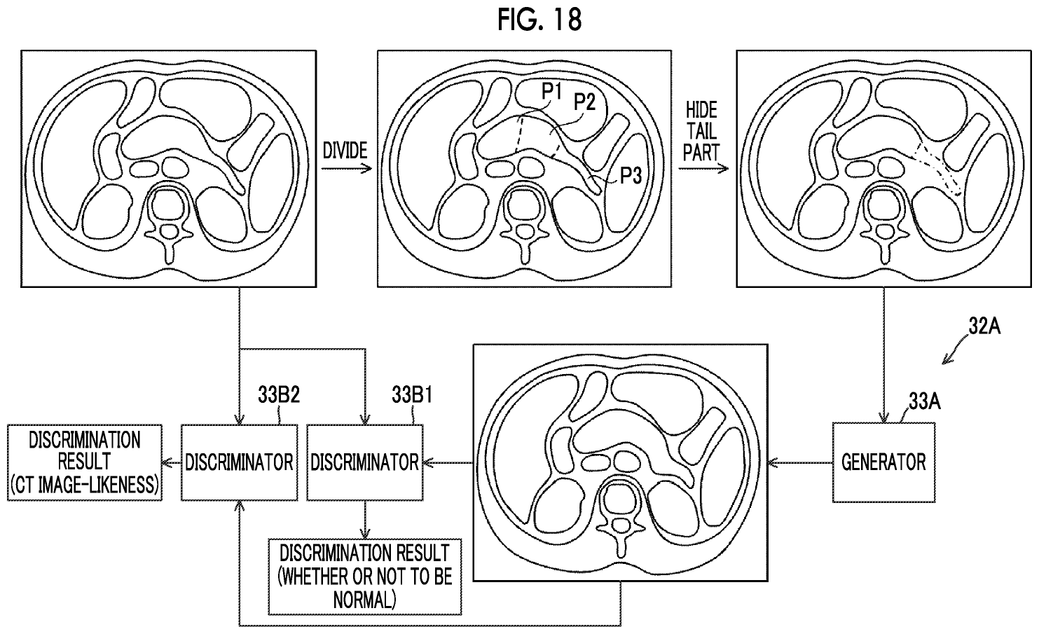

In addition, as shown in FIG. 18 as an example, the trained model 32 may comprise two discriminators 33B1 and 33B2. In this case, for example, the discriminator 33B1 discriminates whether or not a shape of the pancreas in the estimated medical image is normal by comparing the medical image in which an abnormality has occurred in the pancreas with the estimated medical image output from the generator 33A. In addition, in this case, the discriminator 33B2 discriminates whether the estimated medical image is likely to be a CT image by comparing the medical image in which an abnormality has occurred in the pancreas with the estimated medical image output from the generator 33A. The CT image-likeness can be discriminated, for example, from a statistical value of the CT value.

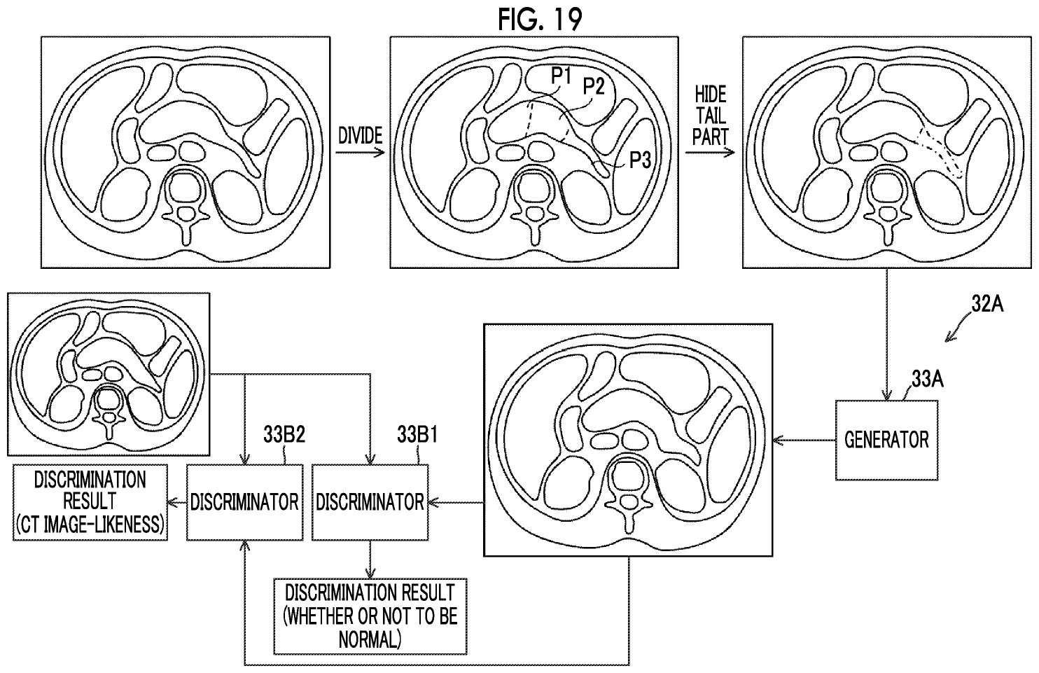

In addition, as shown in FIG. 19 as an example, in this embodiment example, the discriminator 33B1 and the discriminator 33B2 may compare the normal medical image with the estimated medical image. The normal medical image in this case and the medical image in which an abnormality has occurred in the pancreas may be obtained by imaging the same patient or may be obtained by imaging different patients.

In addition, in each of the above embodiments, a case in which the display controllers 60 and 60A perform control of displaying the partial region as the estimation target in the estimated medical image and the partial region as the estimation target in the diagnosis target image in a comparable manner has been described, but the present disclosure is not limited to this. The display controllers 60 and 60A may perform control of displaying information indicating a difference between the partial region as the estimation target in the estimated medical image and a region corresponding to the partial region as the estimation target in the diagnosis target image.

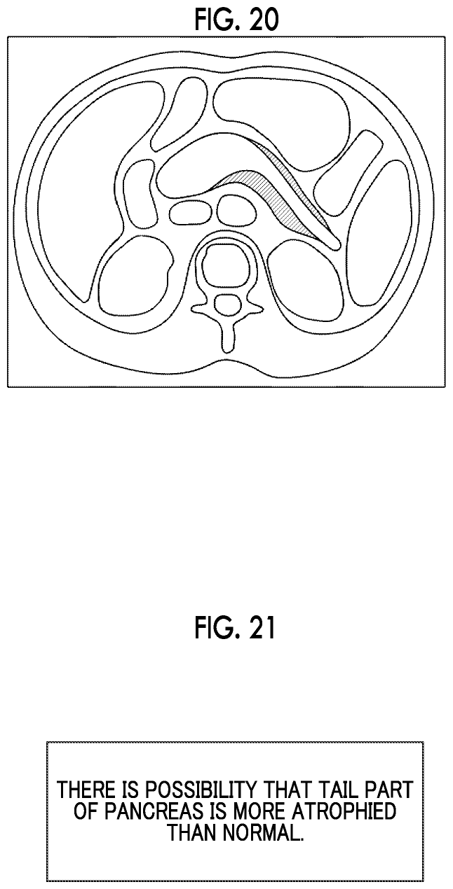

For example, as shown in FIG. 20, the display controllers 60 and 60A may perform control of displaying the partial region as the estimation target in the estimated medical image and the region corresponding to the partial region as the estimation target in the diagnosis target image in a superimposed manner in a state of filling a region of a difference between the partial regions with a predetermined color. In the example of FIG. 20, a region of a difference between the pancreas of the estimated medical image and the pancreas of the diagnosis target image is filled with diagonal lines. In addition, the display controllers 60 and 60A may change a color of the region of the difference depending on a value indicating the difference. Specifically, for example, the display controllers 60 and 60A may cause each voxel in the region of the difference to have a color closer to blue as the difference in the CT value is smaller, and cause each voxel in the region of the difference to have a color closer to red as the difference in the CT value is larger.

In addition, for example, the display controllers 60 and 60A may perform control of displaying a contour of the partial region as the estimation target in the estimated medical image and a contour of the region corresponding to the partial region as the estimation target in the diagnosis target image in a superimposed manner.

In addition, for example, the display controllers 60 and 60A may generate an image using volume rendering or surface rendering for each of the partial region as the estimation target in the estimated medical image and the partial region as the estimation target in the diagnosis target image. In this case, the display controllers 60 and 60A may perform control of displaying the generated images side by side or control of displaying the generated images in a superimposed manner.

In addition, for example, as shown in FIG. 21, the display controllers 60 and 60A may perform control of displaying a text indicating a difference between the partial region as the estimation target in the estimated medical image and the region corresponding to the partial region as the estimation target in the diagnosis target image. FIG. 21 shows an example of a text in a case in which the tail part P3 of the pancreas in the diagnosis target image is atrophied.

In addition, in each of the above embodiments, a case in which the pancreas is applied as the anatomical region to be processed, and the head part, the body part, and the tail part are applied as the plurality of partial regions in the anatomical region has been described, but the present disclosure is not limited to this. For example, the liver may be applied as the anatomical region to be processed, and each region such as SI to S8 may be applied as the plurality of partial regions in the anatomical region. In addition, for example, the small intestine may be applied as the anatomical region to be processed, and the duodenum, the jejunum, and the ileum may be applied as the plurality of partial regions in the anatomical region. In addition, for example, the pancreas may be applied as the anatomical region to be processed, and the pancreatic parenchyma and the pancreatic duct may be applied as the plurality of partial regions in the anatomical region.

In addition, in each of the above embodiments, as the trained model 32, a model that generates an estimated medical image in which the body part P2 is estimated based on the head part P1 may be applied, or a model that generates an estimated medical image in which the tail part P3 is estimated based on the head part P1 may be applied. In addition, as the trained model 32, a model that generates an estimated medical image in which the body part P2 and the tail part P3 are estimated based on the head part P1 may be applied.

In addition, in each of the above embodiments, as the trained model 32, a model that generates an estimated medical image in which the head part P1 is estimated based on the body part P2 may be applied, or a model that generates an estimated medical image in which the tail part P3 is estimated based on the body part P2 may be applied. In addition, as the trained model 32, a model that generates an estimated medical image in which the head part P1 and the tail part P3 are estimated based on the body part P2 may be applied.

In addition, in each of the above embodiments, as the trained model 32, a model that generates an estimated medical image in which the head part P1 is estimated based on the tail part P3 may be applied, or a model that generates an estimated medical image in which the body part P2 is estimated based on the tail part P3 may be applied. In addition, as the trained model 32, a model that generates an estimated medical image in which the head part P1 and the body part P2 are estimated based on the tail part P3 may be applied.

In addition, in each of the above embodiments, the estimated medical image of the pancreas in which the head part P1, the body part P2, and the tail part P3 estimated by the trained models 32A, 32B, and 32C are combined may be generated.

In each of the above embodiments, a case in which the trained model 32 is configured by the CNN has been described, but the present disclosure is not limited to this. The trained model 32 may be configured by a machine learning method other than the CNN.

In addition, in each of the above embodiments, a case in which a CT image is applied as the diagnosis target image has been described, but the present disclosure is not limited to this. As the diagnosis target image, a medical image other than the CT image, such as a radiation image captured by a simple X-ray imaging apparatus and an MRI image captured by an MRI apparatus, may be applied.

The processes in steps S20 to S28 and S20 to S26A of the diagnosis support process according to the above embodiments may be executed before an instruction to start an execution is input by the user. In this case, in a case in which the user inputs an instruction to start an execution, the processes after step S30 or step S32A is executed, and the screen is displayed.

In addition, in each of the above embodiments, for example, various processors shown below can be used as a hardware structure of a processing unit that executes various kinds of processing, such as each functional unit of the image processing apparatus 10. The various processors include, as described above, in addition to a CPU, which is a general-purpose processor that functions as various processing units by executing software (program), a programmable logic device (PLD) that is a processor of which a circuit configuration may be changed after manufacture, such as a field programmable gate array (FPGA), and a dedicated electrical circuit which is a processor having a circuit configuration specially designed to execute specific processing, such as an application specific integrated circuit (ASIC).

One processing unit may be configured of one of the various processors, or may be configured of a combination of the same or different kinds of two or more processors (for example, a combination of a plurality of FPGAs or a combination of the CPU and the FPGA). In addition, a plurality of processing units may be configured of one processor.

As an example in which a plurality of processing units are configured of one processor, first, as typified by a computer such as a client or a server, there is an aspect in which one processor is configured of a combination of one or more CPUs and software, and this processor functions as a plurality of processing units. Second, as typified by a system on chip (SoC) or the like, there is an aspect in which a processor that implements functions of the entire system including the plurality of processing units via one integrated circuit (IC) chip is used. As described above, various processing units are configured by using one or more of the various processors as a hardware structure.

Further, as the hardware structure of the various processors, more specifically, an electric circuit (circuitry) in which circuit elements such as semiconductor elements are combined may be used.

In addition, in the above embodiment, an aspect has been described in which the learning program 30 and the image processing program 31 are stored (installed) in the storage unit 22 in advance, but the present disclosure is not limited to this. The learning program 30 and the image processing program 31 may be provided in a form of being recorded in a recording medium, such as a compact disc read only memory (CD-ROM), a digital versatile disc read only memory (DVD-ROM), and a universal serial bus (USB) memory. In addition, the learning program 30 and the image processing program 31 may be downloaded from an external device via the network.

What is claimed is:

1. An image processing apparatus comprising:
at least one processor,
wherein the processor generates an estimated medical image in which at least one partial region as an estimation target among a plurality of partial regions in an anatomical region included in a medical image is estimated based on at least one partial region other than the estimation target.

2. The image processing apparatus according to claim 1,
wherein the processor divides the anatomical region into the plurality of partial regions.

3. The image processing apparatus according to claim 1,
wherein the processor performs control of displaying the partial region as the estimation target in the estimated medical image and a region corresponding to the partial region as the estimation target in the medical image in a comparable manner.

4. The image processing apparatus according to claim 1,
wherein the processor performs control of displaying information indicating a difference between the partial region as the estimation target in the estimated medical image and a region corresponding to the partial region as the estimation target in the medical image.

5. The image processing apparatus according to claim 3,
wherein the processor performs the control in a case in which a value indicating a difference between the partial region as the estimation target in the estimated medical image and the region corresponding to the partial region as the estimation target in the medical image is equal to or greater than a threshold value.

6. The image processing apparatus according to claim 5,
wherein the processor
generates the estimated medical image for each of the plurality of partial regions, and
performs the control in a case in which a value indicating the difference for at least one estimated medical image is equal to or greater than the threshold value.

7. The image processing apparatus according to claim 1,
wherein the estimated medical image is an image in which the estimated medical image generated for at least one of the plurality of partial regions is combined with the anatomical region in the medical image.

8. The image processing apparatus according to claim 1,
wherein the processor
performs a process of detecting a candidate for an abnormality in the anatomical region, and
generates the estimated medical image using only a trained model corresponding to the partial region in which the detected candidate for the abnormality exists among a plurality of trained models that are respectively trained in advance for the plurality of partial regions, the trained model being used to generate the estimated medical image.

9. The image processing apparatus according to claim 1,
wherein the anatomical region is a pancreas, and
the plurality of partial regions include a head part, a body part, and a tail part.

10. An image processing method executed by a processor of an image processing apparatus, the method comprising:
generating an estimated medical image in which at least one partial region as an estimation target among a plurality of partial regions in an anatomical region included in a medical image is estimated based on at least one partial region other than the estimation target.

11. A non-transitory computer-readable storage medium storing an image processing program for causing a processor of an image processing apparatus to execute:
generating an estimated medical image in which at least one partial region as an estimation target among a plurality of partial regions in an anatomical region included in a medical image is estimated based on at least one partial region other than the estimation target.

12. A learning apparatus comprising:
at least one processor,
wherein the processor performs machine learning using an estimated medical image in which at least one first partial region as an estimation target among a plurality of partial regions in an anatomical region included in a medical image is estimated based on at least one second partial region other than the first partial region, and a normal medical image in which an abnormality has not occurred in the anatomical region, as learning data, thereby generating a trained model that outputs the estimated medical image in response to an input of the second partial region.

13. A learning method executed by a processor of a learning apparatus, the method comprising:
performing machine learning using an estimated medical image in which at least one first partial region as an estimation target among a plurality of partial regions in an anatomical region included in a medical image is estimated based on at least one second partial region other than the first partial region, and a normal medical image in which an abnormality has not occurred in the anatomical region, as learning data, thereby generating a trained model that outputs the estimated medical image in response to an input of the second partial region.

14. A non-transitory computer-readable storage medium storing a learning program for causing a processor of a learning apparatus to execute:
performing machine learning using an estimated medical image in which at least one first partial region as an estimation target among a plurality of partial regions in an anatomical region included in a medical image is estimated based on at least one second partial region other than the first partial region, and a normal medical image in which an abnormality has not occurred in the anatomical region, as learning data, thereby generating a trained model that outputs the estimated medical image in response to an input of the second partial region.
